(19) United States
(12) Patent Application Publication
(10) Pub. No.: US 2015/0050661 A1
(43) Pub. Date: Feb. 19, 2015

(54) SYSTEM AND METHOD FOR MONITORING AND OPTIMIZING IMMUNE STATUS IN TRANSPLANT RECIPIENTS

(71) Applicant: Dartmouth-Hitchcock Clinic, Lebanon, NH (US)

(72) Inventors: Richard A. Zuckerman, M.D., Norwich, VT (US); Michael C. Chobanian, Etna, NH (US)

(21) Appl. No.: 14/470,547

(22) Filed: Aug. 27, 2014

Related U.S. Application Data
(63) Continuation of application No. 12/900,450, filed on Oct. 7, 2010, now abandoned.
(60) Provisional application No. 61/249,734, filed on Oct. 8, 2009.

(51) Int. Cl. C12Q 1/68

(52) U.S. Cl. CPC C12Q 1/6876; C12Q 2600/112; C12Q 2600/158
USPC 435/6.12

(57) ABSTRACT

This invention provides a system and method for an assay used in determining appropriate immunosuppressant levels relative to organ transplant in which PBMC is separated from whole blood by Ficoll®. An aliquot of PBMC is used for phenotyping of cells. CD4, CD8, memory and naïve subsets, B-cells regulatory T-cells and other cell markers (e.g. CD31) are examined. After an aliquot of PBMC is taken, CD4 cells are isolated. DNA is isolated from the cells. CD4 cells can be used for TREC at the defined time points. The TREC assay can be performed via a validated protocol. TREC levels are then measured using a quantitative RT-PCR for single jointed TREC. Alternatively, or additionally, TREC-correlated cell markers (e.g. CD31) can be analyzed. Approximately 100,000 cells, or 2 micrograms, of DNA are desired for TREC analysis. Normal control cells are run in parallel. A kit, including instructions and various components can be provided.

### RELATED APPLICATION

This application is a continuation of U.S. patent application Ser. No. 12/900,450, filed Oct. 7, 2010, entitled SYSTEM AND METHOD FOR APPLYING AN ASSAY FOR DETECTION AND QUANTIFICATION OF T-CELL RECEPTOR EXCISION CIRCLES IN TRANSPLANT RECIPIENTS, which claims the benefit of U.S. Provisional Application Ser. No. 61/249,734, filed Oct. 8, 2009, entitled SYSTEM AND METHOD FOR APPLYING AN ASSAY FOR DETECTION AND QUANTIFICATION OF T-CELL RECEPTOR EXCISION CIRCLES IN TRANSPLANT RECIPIENTS, the entire disclosure of each of which applications is herein incorporated by reference.

### TECHNICAL FIELD

This invention relates, in general, to systems and methods for optimizing the level of immune competence (or status) in transplant recipients.

### BACKGROUND

Transplantation of solid organs is currently the treatment of choice for all patients with end stage organ failure involving the kidney, liver, lungs and heart. New and novel means of inducing and maintaining those organs in patients has led to marked improvements in both patient and organ survival. The end result of these therapies is a net state of immune suppression, which if too severe, is highly associated with infection, morbidity, organ loss and death. Currently, most organ recipients receive similar medications and doses after transplant with the expectation that they will be tailored based on the level of the medication and/or side effects, including infection and rejection. This practice is inherently imperfect in that alterations usually occur after adverse sequellae. To date, a reliable predictor of immune competence or status (i.e. the relative ability of the patient's immune system to respond to antigens) in transplant has been elusive.

More than 80 percent of the world's transplant centers utilize induction therapy (in general, the use of very potent medications at the time of transplant, typically given intravenously during and immediately following the transplant procedure) to prepare a transplant patient/recipient for transplantation of solid organs. The potency of this induction therapy is such that, when taken together with the maintenance therapy (often involving the use of oral immunosuppressant medications daily to continually suppress immune system so as to help prevent rejection required post-transplant), is such that it renders the patient immune incompetent. Because of the effects of the potency of this form of immunosuppression, the rates and severity of infections, malignancies and other side effects in the patient have increased. Overall, the complications resulting for such immunosuppression therapy require that the patient's immune state be accurately and continuously monitored.

After ablation with induction therapy, the patient's lymphocytes are driven to repopulate the periphery. In the presence of an intact thymus, many of these new cells may originate from the thymus as naïve cells. However, without residual thymus, the majority of cells that repopulate the periphery will be derived from peripheral cells of a memory phenotype, a process called homeostatic repopulation. An imbalance of naïve and memory cells may be responsible for the negative outcomes after transplantation and the need for additional immunotherapeutics. For example, it is felt that effector memory cells originating from homeostatic repopulation of lymphocytes are responsible for rejection. Because each individual patient enters transplantation with a different repertoire of immune cells, it only makes sense that the same immunosuppressive medications will affect each patient differently.

Regulatory T-cells play an important role in immune homeostasis and are felt to be important in organ tolerance in the transplant setting. Nevertheless, recent research has been inconclusive with regard to the role of regulatory T-cells in the setting of rejection and tolerance. Some of this discrepancy may be attributed to the origin of the regulatory T-cells after transplantation. For example, because induction is followed by homeostatic proliferation of T-cells, including regulatory T-cells, the function of these cells is likely different than regulatory T-cells that are derived from naïve cells in the thymus.

Recent advances in molecular technology have enabled researchers to quantitatively evaluate peripheral blood cells for the presence of cells that have recently emigrated from the thymus, the origin of most peripheral T-lymphocytes. Circular DNA residues, called T-cell receptor excision rearrangement circles (TRECs) are present only in cells recently emigrating from the thymus and can be quantified in a polymerase chain reaction (PCR). While this technology has been used to show age-related thymic involution and the dynamics of immune reconstitution after stem cell transplantation and in HIV. By optimizing thymic output in a transplant procedure, the number of naïve cells is improved post-transplant. This broadens the immune system's ability to respond to novel pathogens, and more significantly, can potentially enhance the ability to generate organ tolerance. This leads to a greater likelihood of non-rejection of the transplanted organ throughout a range of transplant recipients. This is a key goal in organ transplantation.

It is, therefore, desirable to provide a reliable predictor of immune competence in transplant. Additionally, a method to predict and monitor levels of immune suppression after transplantation is also desirable. Further, there is a desire to design essential immunosuppression for recipients of solid organ transplants at specific ages.

### SUMMARY

This invention overcomes the disadvantages of the prior art by utilizing a molecular assay to show that the immune repertoire after solid organ transplantation is directly correlated with pre-transplant thymic activity. Additionally, the amount of residual thymic activity prior to transplant will predict the types and function of cells that expand thereafter.

It is desirable according to an illustrative embodiment of the present invention to provide an assay or other diagnostic modality in which TREC measurements and other cell markers (e.g. CD31 surface marker that correlates with thymic migrants), which are correlated to TREC measurements can be used pre-transplant as markers of T-cell competency in potential solid organ transplant recipients and guide immunosuppressive induction and maintenance schemes. It is further desirable of the present invention to provide an assay in which TREC and/or other cell marker(s) (e.g. CD31) determinations post-transplant can identify an individual's need and response to specific immunosuppressives, and to provide an assay in which TREC and/or other cell marker(s) measurements can lead to decreased acute complications such as post-transplant infections and chronic complications such as post-transplant cancers.

An illustrative embodiment can provide an assay that will define the relationship of TREC to the kinetics of repopulation of TREC, regulatory T-cells and other cell subsets after kidney transplantation, and can also provide an assay that will show that thymic reserves correlate directly with immune reconstitution and clinical outcomes after transplantation. Illustratively, TREC and/or other cell markers is/are employed to guide immunosuppression protocols.

Illustratively, an assay is provided for determination of TREC levels. In this assay, PBMC is separated from whole blood by Ficoll. An aliquot of PBMC is used for phenotyping of cells. CD4, CD8, memory and naïve subsets, B-cells, regulatory T-cells and other cell markers (e.g. CD31) are examined. After an aliquot of PBMC is taken, CD4 cells are isolated. In one example, this is accomplished by negative selection using a Robosep Magnetic Bead Sorter isolating CD4+ cells. An alternate method for sorting can be, for example, positive selection. CD4 cells are used for TREC at the defined time points. The TREC assay is performed via the validated Duke University protocol. Briefly, DNA is isolated from cells using a Gentra PureGene blood kit or another acceptable blood kit. TREC levels are then measured using a quantitative RT-PCR for single jointed TREC (sjTREC) using an AB 7500 FAST System. Approximately 100,000 cells, or 2 micrograms, of DNA are required for TREC analysis. Blood volume requirements are tailored to ensure enough DNA for TREC analysis is obtained. Normal control cells are run in parallel.

In an illustrative embodiment, a medical treatment method for determining and controlling of immune status in a transplant patient includes separating cellular components that correlate with patient TREC levels, analyzing the cellular components to determine the patient TREC levels, and controlling immunosuppressant administration to the patient based upon the determined TREC levels. The step of analyzing the cellular components can further include analyzing TREC surrogates. These TREC surrogates can include cell markers, such as CD31.

In an illustrative embodiment, the instructions and various components/compounds of the assay and/or other compounds, needed to carry out a TREC analysis and/or analysis (e.g. flow) for other cell markers for the purpose of determining a patient/recipient's immune status can be provided in a kit available to the practitioner in the art.

### BRIEF DESCRIPTION OF THE DRAWINGS

The invention description below refers to the accompanying drawings, of which:

FIG. 1 is a block diagram showing the assay method of the present invention; and

FIG. 2 is a block diagram showing a generalized interaction involving the employment of a kit for accomplishing the system and method according to the illustrative embodiment.

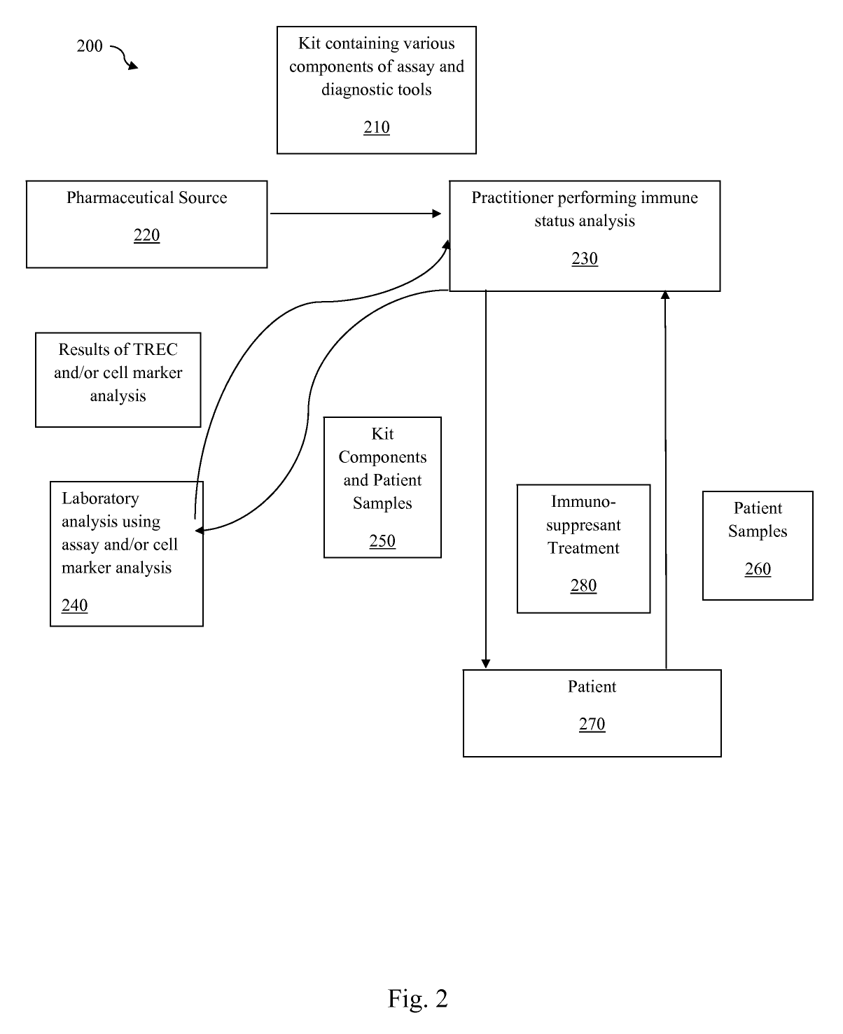

### DETAILED DESCRIPTION

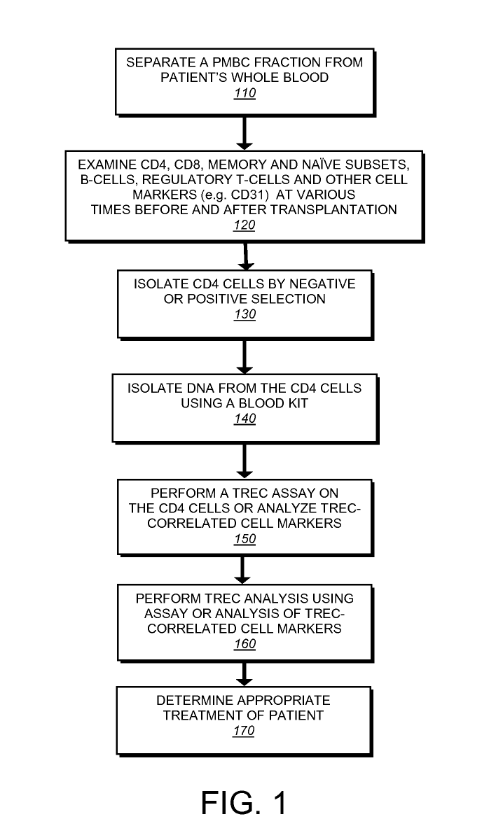

FIG. 1 details an analysis procedure for determining the immune status and appropriate treatment for a solid organ transplant patient using an assay to be described below. As used herein the term “solid organ” should be taken broadly to include, at least bone marrow, and can include other forms of transplantable tissue in which the patient's well being and physiological non-rejection of the transplant would be aided by the ability to monitor immune status within the teachings of this system and method. The term “transplant” or “transplantation” as used herein should be taken as any procedure that introduces foreign organs, or tissue to a patient/recipient's body in which there is a risk of an immune response that can lead to rejection or another undesired physiological response, and that requires, for example, induction and maintenance therapy so as to avoid such undesired response.

In the assay and in accordance with the method 100 of FIG. 1, a peripheral blood mononuclear cell (PBMC) is separated from whole blood by Ficoll® (Step 110), thereby constituting the PMBC fraction of the whole blood. An exemplary procedure for doing so (see Ficoll-Hypaque PMBC Separation, Rev: J. Hale Oct. 19, 2005) is as follows—however, it is expressly contemplated that any of the compounds, steps and/or methodologies described herein can be substituted for other compounds, steps and/or methodologies that achieve equivalent results:

1. Collect heparinized blood. 2. Dilute whole blood 1 to 1 with sterile saline (0.9% NaCl) and mix. 3. Layer up to 35 mL Blood/Saline mixture over 13 mL LSM in as many 50 mL conical tubes as necessary.
Spin@1,250 rpm for 35 min, room temp, brake off. 4. Aspirate and discard plasma top layer.
Collect the PBMC buffy coat above the clear LSM Layer.
Combine cells from two tubes into one 50 mL conical tube.
Bring to 50 mL total volume with RPMI.
Spin@1,800 rpm for 8 min, room temp.

5. Decant supernatant and discard.
Resuspend in 10 mL RPMI and mix.
Spin@1500 rpm for 5 min, 4° C.

6. Decant supernatant and discard.
Resuspend in 10 mL RPMI and mix.
Count cells on Coulter Counter using 40 uL of cell suspension
X cells/mL×10 mL=Y total cells
Spin@1,500 rpm for 5 min, 4° C.

7. Decant supernatant and discard.
Resuspend to desired concentration in aliquots of desired medium.
   a) 90% RPMT+10% FBS+Gent (5 ug/mL) for immediate use OR
   b) 90% Human AB serum—heat inactivated+10% DMSO for cryopreserve (10-20 million cells/mL) OR
   c) MACS Buffer for bead isolation.

It is contemplated that phenotyping of cells can occur in accordance with various embodiments of the invention. In an illustrative embodiment, whole blood staining is employed in accordance with a conventional implementation. In alternate embodiments, alternate technique van be employed, including, for example, using an aliquot of peripheral blood mononuclear cells (PBMC).

CD4, CD8, memory and naïve subsets, B-cells, regulatory T-cells and other appropriate cell markers will be examined at various time points before and after transplantation (Step 120). Illustratively the term “cell markers” shall refer to any marker, such as the surface marker CD31 that is, or is shown to be, correlated with TREC levels. In step 130, after an aliquot of PBMC is taken, CD4+ cells are isolated by negative or positive selection—for example using a Robosep Magnetic Bead Sorter or Stem Cell separation. The procedure for doing so (see Miltenyi MACS Positive Bead Separation, Rev: J. Hale Oct. 19, 2005) can be as follows (using an exemplary Robosep Magnetic Bead Sorter):

1. Place cells in 15 mL conical tube.
Add 5 mL of MACS buffer.

2. Spin@1,500 rpm for 5 min at 4° C.
Aspirate with Pasteur pipette attached to vacuum or through manual pipetting.

3. Resuspend in 80 uL of MACS buffer and 20 uL of vortexed Miltenyi beads per 10⁷ total cells present. Use a min of 80 uL of MACS buffer and 20 uL of beads.
Mix with pipette and vortex.
Incubate at 4° C. for 15 min.

4. Place MS columns in OctoMACS magnet (use one column per sample per bead type).
Wash each column with 3 mL MACS buffer. Collect the flow through in waste tubes.

5. Add 5 mL MACS buffer to the cells.
Spin @2,000 rpm for 10 min at 4° C.
Aspirate.
Resuspend in 0.500 mL MACS buffer.
Place a labeled 15 mL tube below the column to collect negative fraction for any additional separation.
Add cells to column. (Pass cells through 70 um filter to remove clumps.)

6. Rinse columns 3× with 0.500 mL of MACS buffer. Continue to collect the flow through. Use a 2^{nd} column and combine the results, if the cells refuse to flow through the column.

7. Remove columns from magnet.
Place in fresh labeled 15 mL tube.
Let rest 5 min.

8. Add 2 mL MACS buffer to the column.
Plunge through once to recover the positive fraction.
Discard the column.
Place positive fraction on ice.

Repeat Steps 2 through 8 for each type of separation desired using different beads and the desired fraction.

9. Spin fractions @1,500 rpm for 5 min at 4° C.
Aspirate.
Resuspend in 1 mL of MACS buffer.

Briefly, DNA is isolated from cells using an appropriate blood kit ( )Step 150). Where the blood kit does not potentially damage separated T-cells, it can be employed in the lysis step (step 1 below). By way of example, a Gentra PureGene blood kit (see Gentra® Puregene® Handbook, Second Edition, September 2007) is used and is safely employed at step 4, or later, below. In an illustrative procedure, one of three choices can be made for some steps of the procedure depending upon the size of the blood sample. Choose ▪ if processing 300 μl blood samples; choose ▴ if processing 3 ml blood samples; choose if processing 10 ml blood samples. The remaining exemplary procedure is as follows:

1. Dispense ▪ 900 μl, ▴ 9 ml, or 30 ml RBC Lysis Solution into a ▪ 1.5 ml microcentrifuge tube, ▴ 15 ml centrifuge tube, or 50 ml centrifuge tube.

2. Add ▪ 300 μl, ▴ 3 ml, or 10 ml whole blood or bone marrow, and mix by inverting 10 times.

3. Incubate ▪ 1 min, ▴ 5 min, or 5 min at room temperature (15-25° C.). Invert at least once during the incubation.

▪ For fresh blood (collected within 1 h before starting the protocol), increase incubation time to 3 min to ensure complete red blood cell lysis.

4. Centrifuge for ▪ 20 s at 13,000-16,000×g, ▴ 2 min at 2000×g, or 2 min at 2000×g to pellet the white blood cells.

5. Carefully discard the supernatant by pipetting or pouring, leaving approximately ▪ 10 μl, ▴ 200 μl, or 200 μl of the residual liquid and the white blood cell pellet.

6. Vortex the tube vigorously to resuspend the pellet in the residual liquid. Vortexing greatly facilitates cell lysis in the next step. The pellet should be completely dispersed after vortexing.

7. Add ▪ 300 μl, ▴ 3 ml, or 10 ml Cell Lysis Solution, and pipet up and down to lyse the cells or vortex vigorously for 10 s. Usually no incubation is required; however, if cell clumps are visible, incubate at 37° C. until the solution is homogeneous. Samples are stable in Cell Lysis Solution for at least 2 years at room temperature.

8. Optional: If RNA-free DNA is required, add ▪ 1.5 μl, ▴ 15 μl, or 50 μl RNaseA Solution, and mix by inverting 25 times. Incubate for 15 min at 37° C. Then incubate for ▪ 1 min, ▴ 3 min, or 3 min on ice to quickly cool the sample.

9. Add ▪ 100 μl, ▴ 1 ml, or 3.33 ml Protein Precipitation Solution, and vortex vigorously for 20 s at high speed.

10. Centrifuge for ▪ 1 min at 13,000-16,000×g, ▴ 5 min at 2000×g, or 5 min at 2000×g. The precipitated proteins should form a tight, dark brown pellet. If the protein pellet is not tight, incubate on ice for 5 min and repeat the centrifugation.

11. Pipet ▪ 300 μl isopropanol into a clean 1.5 ml tube, ▴ 3 ml isopropanol into a clean 15 ml tube, or 10 ml isopropanol into a clean 50 ml tube and add the supernatant from the previous step by pouring carefully. Be sure the protein pellet is not dislodged during pouring.

12. Mix by inverting gently 50 times until the DNA is visible as threads or a clump.

13. Centrifuge for ▪ 1 min at 13,000-16,000×g, ▴ 3 min at 2000×g, or 3 min at 2000×g. The DNA may be visible as a small white pellet.

14. Carefully discard the supernatant, and drain the tube by inverting on a clean piece of absorbent paper, taking care that the pellet remains in the tube.

15. Add ▪ 300 μl, ▴ 3 ml, or 10 ml of 70% ethanol and invert several times to wash the DNA pellet.

16. Centrifuge for ▪ 1 min at 13,000-16,000×g, ▴ 1 min at 2000×g, or 1 min at 2000×g.

17. Carefully discard the supernatant. Drain the tube on a clean piece of absorbent paper, taking care that the pellet remains in the tube. Air dry the pellet for ▪ 5 s, ▴ 1 min, or 10-15 min. The pellet might be loose and easily dislodged. Avoid over-drying the DNA pellet, as the DNA will be difficult to dissolve.

18. Add ▪ 100 μl, ▴ 250 μl, or 1 ml DNA Hydration Solution and vortex for 5 s at medium speed to mix.

19. Incubate at 65° C. for ▪ 5 min, ▴ 1 h, or 1 h to dissolve the DNA.

20. Incubate at room temperature overnight with gentle shaking Ensure tube cap is tightly closed to avoid leakage. Samples can then be centrifuged briefly and transferred to a storage tube.

CD4 cells are illustratively used for TREC at the defined time points (Step 150). The TREC assay (for example, as described in U.S. Pat. No. 6,544,747), which is expressly incorporated herein by reference, is illustratively performed via the validated Duke University protocol (see TREC PCR (Human or Mouse) Rev: J. Hale Oct. 19, 2005), which is as follows:

1. Obtain PCR reagents from PCR hood.
Thaw at 56° C. for 2-3 min.
Clean p20, p200, and p1000 with ethanol.
Thaw samples on bench
Change gloves

2. Mix appropriate amounts of PCR reagents for the desired number of wells in Eppendorf tube or 15 mL conical tube.

| PCR Mix | uL per well |
|---|---|
| Platinum Taq Buffer | 2.500 |
| 50 mM MgCl₂ | 1.750 |
| 10 mM dNTP | 0.500 |
| 12.5 uM 5′ primer | 1.000 |
| 12.5 uM 3′ primer | 1.000 |
| 5 uM probe | 1.000 |
| Platinum Taq | 0.125 |
| Water, PCR grade | 12.125 |
| Vortex PCR mix. | |

3. Add 20 uL of PCR Mix to each well (standards, samples, and NTC).

4. Add 5 uL of water to NTC and cap.
Vortex samples
Spin samples

5. Add 5 uL of sample (50,000 cell equivalents or 1 ug DNA) to appropriate well in duplicate. Cap every row as completed.
Freeze remaining sample. Remove PCR reagents from UV hood.
UV light the PCR hood for several minutes
Change gloves
Work on clean bench using Standards-only rack, caps, tips, and pipette.

6. Thaw standards at room temperature 2-3 minutes
Add 5 uL standards to appropriate wells, in duplicate, from lowest to highest (˜10² to ˜10⁷)
Cap standards

7. Shake plate
Briefly centrifuge plate

8. Place plate in PCR machine.

9. Report 2× the number of TRECs given in the results to get # of TRECs/100,000 cells or report #TREC/lug DNA.

In accordance with step 160, the TREC analysis is then performed based upon the assay. Alternatively (or in addition) an analysis using TREC-correlated cell markers can be performed. In an exemplary PCR methodology (using an acceptable PCR device and/or procedure known to those of skill in the art), TREC levels are then measured using a quantitative RT-PCR for single jointed TREC (sjTREC). Using primers directed against the sjTREC sequence, the polymerase chain reaction (PCR) is used to amplify this segment of DNA. The PCR occurs by ramping between temperatures for denaturation, annealing and extension of DNA and results in millions of copies of the original target sequence. This then allows for ample material to be quantified by using similar amplification of known concentrations of target DNA.

Notably, in research reported subsequent to the filing of the above-incorporated co-pending U.S. Provisional Application Ser. No. 61/249,734, the described connection between immunosuppresant treatment and TREC levels in said provisional application have been validated. See Ducloux, et al., Prolonged CD4 T Cell Lymphopenia Increases Morbidity and Mortality after Renal Transplantation, J. Am. Soc. Nephrol 21: 868-875, May 2010. Thus, the novel treatment techniques and treatment kit described herein is further shown to be a valid approach.

To determine TREC levels, calibration curves are created for each assay by plotting cycle threshold (Ct) values detected during the PCR against the concentrations in a dilution series of a known concentration of a plasmid containing the sjTREC target. TREC levels are reported as the number of TREC per 100,000 cells.

The TREC analysis described above is employed by the practitioner in determining appropriate treatment for the patient having undergone organ transplantation (step 170).

As described above, alternatively, cell markers that are correlated to TREC (e.g. CD31) are used as a surrogate for TREC measurement. The presence of such cell markers can be monitored using conventional techniques that are readily employed by clinicians. For example, in the case of the CD31 marker, presence and levels can be determined by conventional flow cytometry (FCM) techniques.

The illustrative kit for use by a practitioner/clinician in determining immune status and/or immune competence can provide various data related to, for example, appropriate immunosuppressant levels to be applied and/or information related to the patient's immune status. Illustratively, the procedure and kit can also include protocols in which, after TREC and/or other immune status markers are determined, they are then compared to age-appropriate norms which would be indexed against an “immune competency score.” This score could help the practitioner determine immunosuppressive therapy and dosages. Other scoring metrics can also be employed to influence the score, such as the presence of other medical conditions, patient sex, body mass, etc. Illustratively, the use of clinical trials employing pre-operative and post-operative TREC and/or other cell marker analysis on a patient population can be used to determine common factors that are indicative of appropriate or inappropriate immunosuppressant levels. More generally the illustrative system and method is highly useful in guiding immunosuppressant therapy to thereby optimize an ability to respond to infection and to generally produce organ tolerance within the patient/recipient.

In accordance with an embodiment, the monitoring of TREC levels can be accomplished directly, employing the illustrative assay procedure, or it can be accomplished through monitoring of other cell markers using appropriate conventional techniques (FCM, etc.). More generally, the monitoring of TREC and TREC correlated-cell markers is expressly contemplated to determine immune status and immune competence for the purpose of immunosuppressant regulation. In various embodiments, the determination of TREC levels directly and through use of cell markers can be combined. By way of example, an initial TREC level for the patient can be determined as a baseline, followed by efficient monitoring of cell markers.

Various components employed in the use of the assay to determine appropriate immunosuppressant levels in a post-operative transplant patient according to an illustrative embodiment can be provided in an associated kit 210 as shown in the basic schematic diagram 200 of an exemplary treatment procedure according to the system and method. The kit 210 is provided by a pharmaceutical source or other appropriate entity 220 a practitioner 230 or his/her laboratory 240. The needed components 250, as described herein are employed to perform TREC analysis (directly via the assay or through analysis of TREC-correlated cell markers) based upon samples 260 obtained from a patient 270 in response to application of immunosuppressant treatment 280. The laboratory 240 provides ongoing results 290 that are used to monitor and vary the patient's immunosuppressant levels. Among other components, the kit will include detailed instructional material relating to approved techniques for performing the procedures described herein.

The foregoing has been a detailed description of illustrative embodiments of the invention. Various modifications and additions can be made without departing from the spirit and scope if this invention. Each of the various embodiments described above may be combined with other described embodiments in order to provide multiple features. Furthermore, while the foregoing describes a number of separate embodiments of the apparatus and method of the present invention, what has been described herein is merely illustrative of the application of the principles of the present invention. For example, the principles described herein can be applied to other treatment scenarios, such as those involving blood-based diseases. More particularly, the term TREC analysis” or “analyzing TREC” shall specifically contemplate an analysis of TREC surrogates, such as the above-described cell markers (e.g. CD31). Accordingly, this description is meant to be taken only by way of example, and not to otherwise limit the scope of this invention.

What is claimed is:

1. A kit for guiding immunosuppressant delivery in an organ transplant recipient comprising instructions and components for a user to:
(a) separate a peripheral blood mononuclear cell from whole blood;
(b) examining, with an assay, at least one of CD4, CD8, memory and naïve subsets, B-cells, regulatory T-cells and other cell markers;
(c) isolate DNA of CD4 cells;
(d) generating an assay to perform a TREC analysis on the CD4 cells;
(e) determine immune status of the organ transplant recipient; and
(f) guide immunosuppressant therapy applied to the recipient to thereby optimize an ability to respond to infection and produce organ tolerance.

2. The kit of claim 1 including instructions and components for a user to analyze TREC-correlated cell-markers.

3. The kit of claim 2 further comprising instructions and components for a user to measure TREC levels based upon at least one of (a) the assay and (b) an analysis of cell markers prior to organ transplant so as to provide a marker of immune competency in the organ transplant recipient.

4. The kit of claim 2 further comprising instructions and components for a user to measure TREC levels based upon at least one of (a) the assay and (b) and analysis of cell markers post-transplant so as to identify the organ transplant recipient's need and response to predetermined immunosuppressives.

5. The kit of claim 2 further comprising instructions and components for a user to measure TREC levels based upon at least one of (a) the assay and (b) the analysis of cell markers so as to decrease occurrence of acute complications or chronic complications.

6. The kit of claim 5 wherein the acute complications include post-transplant infections, and the chronic complications include post-transplant cancers.

7. The kit of claim 2 further comprising instructions and components for a user analyze, with the assay, cellular components that include TREC surrogates.

8. The kit of claim 7 wherein the TREC surrogates include CD31 cell markers.

9. The kit of claim 8 wherein the components include predetermined compounds.

10. The kit of claim 2 further comprising instructions and components for a user to compare TREC or other immune status markers to age-appropriate norms, and therefrom define an immune competency score.
